SEQUENCE LISTING (2 sequences)

SEQ ID NO: 1 - protein, 8 aa, Artificial Sequence
  Designed synthetic peptide to act on PI3-Kalpha SH3 domain
RKLPPRSK

SEQ ID NO: 2 - protein, 10 aa, Artificial Sequence
  Designed synthetic peptide to act on PI3-Kalpha SH3 domain
CRKLPPRSKC

(19) United States
(12) Patent Application Publication
(10) Pub. No.: US 2009/0042235 A1
(43) Pub. Date: Feb. 12, 2009

(54) PHOTOREGULATED PEPTIDE, AND METHOD FOR REGULATION OF PEPTIDE-PROTEIN COMPLEX FORMATION USING THE PHOTOREGULATED PEPTIDE

(76) Inventors: Shun Hirota, Nara (JP); Yuji Hamazaki, Kyoto (JP)

Correspondence Address: Cheng Law Group, PLLC, 1100 17th Street, N.W., Suite 503, Washington, DC, 20036, US

(21) Appl. No.: 12/063,569

(22) PCT Filed: Aug. 14, 2006
(86) PCT No.: PCT/JP2006/316278

§ 371 Date: Feb. 11, 2008

(30) Foreign Application Priority Data
Aug. 19, 2005 (JP) 2005-23875

(51) Int. Cl. G01N 33/566; C07K 2/00; C07K 1/00; C07K 1/10; C07K 17/14; C07K 7/02; C07K 7/54; C12Q 1/02; G01N 33/58

(52) U.S. Cl. 435/29; 530/300; 530/333; 530/323

(57) ABSTRACT

The present invention provides a method of photoregulating the formation of a peptide-protein complex by using a novel peptide in which a structural change for recognizing a protein is photoregulated, in the formation of a complex between a peptide capable of recognizing a protein and a protein of interest. A peptide having an intramolecular cross-linkage via a photocleavable cross-linking group, and method for producing the same. Further, a peptide which has an intramolecular cross-linkage via a photocleavable cross-linking group and forms a cyclic structure together with the cross-linking group, containing a peptide having an epitope corresponding to a functional protein or a ligand at a part which forms the cyclic structure, and method for producing the same. A reaction regulation method comprising the steps of: irradiating a peptide which comprises a peptide having the above epitope, with light to cause cleavage of the photocleavable cross-linking group, whereby dissociating the cyclic structure; and initiating a reaction between the peptide in which the cyclic structure has dissociated and the functional protein or the ligand to form a complex.

### TECHNICAL FIELD

The present invention relates to the photoregulation of formation of a peptide-protein complex, and more specifically to a peptide having an intramolecular cross-linkage via a photocleavable cross-linking group, a method for production of the peptide having an intramolecular cross-linkage via a photocleavable cross-linking group, and the photoregulation of formation of a peptide-protein complex using the peptide having an intramolecular cross-linkage via a photocleavable cross-linking group.

### BACKGROUND ART

In general, an active peptide which interacts with a protein lacks photoreactivity.

In the field of structural analysis regarding the folding reaction of a protein, T. Okuno, S. Hirota, and O. Yamauchi, Biochemistry, 39, 7538-7545 (2000) describes the technique of introducing a modifying group which is cleavable by light irradiation into a protein to make the structure of the protein unstable, and irradiating the protein into which the above modifying group is introduced with light to initiate the folding reaction of the protein.

Y. Tatsu, T. Nishigaki, A. Darszon, and N. Yumoto, FEBS Letters, 525, 20-24 (2002) describes an attempt to regulate the formation of a peptide-protein complex by introducing a modifying group which is cleavable by light irradiation into a peptide, and utilizing the fact that the modifying group is cleaved by light irradiation, in the photoregulation of formation of a peptide-protein complex.

However, even when the above modifying group is introduced into a peptide, a structural change in the peptide cannot be regulated, so that it was still difficult to perfectly block recognition of the protein by the peptide. Also, there was a drawback that a bioactive peptide is decomposed by enzyme when it is introduced into the body for use.

### DISCLOSURE OF THE INVENTION

### Object of the Invention

It is an object of the present invention to provide a method of photoregulating the formation of a peptide-protein complex by using a novel peptide in which a structural change for recognizing a protein is photoregulated, in the formation of a complex between a peptide capable of recognizing a protein and a protein of interest.

### SUMMARY OF THE INVENTION

The inventors of the present invention have made diligent efforts for solving the above problems, as a result, and found that the above problems are solved by using a peptide having an intramolecular cross-linkage via a photocleavable cross-linking group, and finally accomplished the present invention.

The present invention includes the following aspects.

(1) A peptide having an intramolecular cross-linkage via a photocleavable cross-linking group.
(2) A peptide which has an intramolecular cross-linkage via a photocleavable cross-linking group and forms a cyclic structure together with the cross-linking group, containing a peptide having an epitope corresponding to a functional protein or a ligand at a part which forms the cyclic structure.
(3) The peptide according to (1) or (2), wherein the photocleavable cross-linking group is the following divalent linking group:

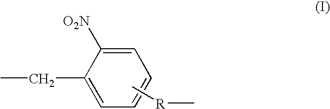

wherein R represents a divalent group.
(4) The peptide according to (1) or (2), wherein the photocleavable cross-linking group is:

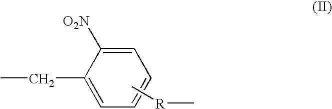

wherein R represents an alkylene group.
(5) The peptide according to (1) or (2), wherein the photocleavable cross-linking group is:

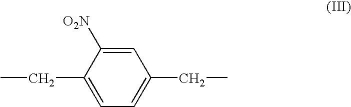

(6) The peptide according to any one of (1) to (5), wherein the photocleavable cross-linking group cross-links between cysteines, between lysines or between cysteine and lysine in the peptide molecule.
(7) The peptide according to any one of (1) to (6), wherein a membrane-permeable peptide is added.
(8) The peptide according to any one of (1) to (7), wherein one end of the peptide is immobilized to a nano bead.
(9) The peptide according to (8), wherein the nano bead is a magnetic bead.
(10) A method for production of the peptide according to (1), wherein side chain functional groups at two positions in a peptide molecule to be cross-linked are subjected to a cross-linking reaction with a compound containing a photocleavable cross-linking group.
(11) The method for production of the peptide according to (10), wherein as the peptide molecule, a peptide molecule containing a peptide having an epitope corresponding to a functional protein or a ligand between the side chain functional groups at the two positions is used.
(12) The method for production of the peptide according to (10) or (11), wherein as the compound containing the photocleavable cross-linking group,

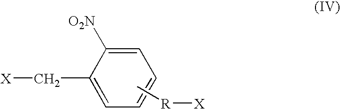

wherein R represents a divalent group and X represents a leaving group or a halogen atom, is used.
(13) The method for production of the peptide according to (10) or (11), wherein as the compound containing the photocleavable cross-linking group,

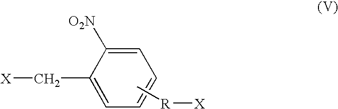

wherein R represents an alkylene group and X represents a leaving group or a halogen atom, is used.
(14) The method for production of the peptide according to (10) or (11), wherein as the compound containing the photocleavable cross-linking group,

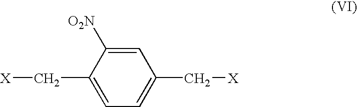

wherein X represents a leaving group or a halogen atom, is used.
(15) The method for production of the peptide according to any one of (10) to (14), wherein the side chain functional groups at two positions are either of:

an SH group and an SH group of cysteines;

an NH₃⁺, group and an NH₃⁺ group of lysines; and

an SH group of cysteine and an NH₃⁺ group of lysine.

(16) The method for production of the peptide according to any one of (10) to (15), wherein a membrane-permeable peptide is added to the peptide obtained by the cross-linking reaction.
(17) The method for production of the peptide according to any one of (10) to (16), wherein the peptide obtained by the cross-linking reaction is immobilized to a nano bead.
(18) The method for production of the peptide according to (17), wherein the nano bead is a magnetic bead.
(19) A reaction regulation method comprising the steps of:

irradiating a peptide which has an intramolecular cross-linkage via a photocleavable cross-linking group and forms a cyclic structure together with the cross-linking group and comprises a peptide having an epitope corresponding to a functional protein or a ligand at a part which forms the cyclic structure, with light to cause cleavage of the photocleavable cross-linking group, whereby dissociating the cyclic structure; and

initiating a reaction between the peptide in which the cyclic structure has dissociated and the functional protein or the ligand to form a complex.

(20) A reaction regulation method comprising the steps of:

administering a peptide which has an intramolecular cross-linkage via a photocleavable cross-linking group and forms a cyclic structure together with the cross-linking group and comprises a peptide having an epitope corresponding to a functional protein or a ligand at a part which forms the cyclic structure, in a biological body;

irradiating the peptide forming the cyclic structure with light to cause cleavage of the photocleavable cross-linking group, whereby dissociating the cyclic structure; and

initiating a reaction between the peptide in which the cyclic structure has dissociated and the functional protein or the ligand to form a complex.

(21) The reaction regulation method according to (19) or (20), wherein the light for the light irradiation is ultraviolet ray.
(22) The reaction regulation method according to any one of (19) to (21), wherein the photocleavable cross-linking group is the following divalent linking group:

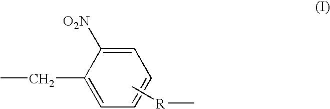

wherein R represents a divalent group.
(23) The reaction regulation method according to any one of (19) to (21), wherein the photocleavable cross-linking group is:

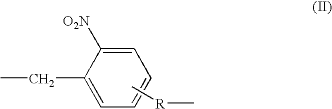

wherein R represents an alkylene group.
(24) The reaction regulation method according to any one of (19) to (21), wherein the photocleavable cross-linking group is:

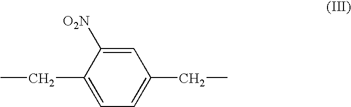

(25) The reaction regulation method according to any one of (19) to (24), wherein the photocleavable cross-linking group cross-links between cysteines, between lysines or between cysteine and lysine in the peptide molecule.
(26) The reaction regulation method according to any one of (19) to (25), wherein a membrane-permeable peptide is added to the peptide which has the intramolecular cross-linkage via the photocleavable cross-linking group and forms the cyclic structure together with the cross-linking group and comprises the peptide having the epitope corresponding to the functional protein or the ligand at the part which forms the cyclic structure.
(27) The reaction regulation method according to any one of (19) to (26), wherein one end of the peptide which has the intramolecular cross-linkage via the photocleavable cross-linking group and forms the cyclic structure together with the cross-linking group and comprises the peptide having the epitope corresponding to the functional protein or the ligand at the part which forms the cyclic structure is immobilized to a nano bead.
(28) The reaction regulation method according to (27), wherein the nano bead is a magnetic bead.
(29) A method comprising the steps of:

immobilizing one end of a peptide which has an intramolecular cross-linkage via a photocleavable cross-linking group and forms a cyclic structure together with the cross-linking group and comprises a peptide having an epitope corresponding to a functional protein or a ligand at a part which forms the cyclic structure, to a magnetic bead;

administering the peptide immobilized to the magnetic bead in a biological body;

irradiating the peptide immobilized to the magnetic bead, with light to cause cleavage of the photocleavable cross-linking group, whereby dissociating the cyclic structure, and reacting the peptide in which the cyclic structure has dissociated with the functional protein or the ligand to form a complex;

collecting the formed complex by a magnet; and

identifying the collected complex by a mass spectrometer.

According to the present invention, by using a peptide having an intramolecular cross-linkage via a photocleavable cross-linking group, it is possible to regulate a structural change in the peptide starting from light irradiation. A peptide capable of recognizing a protein having an intramolecular cross-linkage via a photocleavable cross-linking group forms a cyclic structure together with the photocleavable cross-linking group, so that even in the presence of a protein of interest the peptide is unable to form a tertiary structure for recognizing the protein, and therefore the peptide cannot interact with the protein. However, when the peptide is irradiated with light, the photocleavable cross-linking group is cleaved and the cyclic structure is dissociated, so that the peptide is able to form a tertiary structure for recognizing the protein. As a result, the peptide interacts with the protein and starts the formation of a complex.

As described above, a peptide in which the ring opening of its cyclic structure is regulated by light and thus the formation of a peptide-protein complex is regulated, is a so-called photoregulated peptide.

Further, the peptide having an intramolecular cross-linkage via a photocleavable cross-linking group cannot be degraded easily by enzyme owing to its cyclic structure, so that even when the peptide capable of recognizing a protein is introduced into a biological body a lot of it can be used effectively.

### BRIEF DESCRIPTION OF THE DRAWINGS

FIG. 1 is a view showing an embodiment example of a method for production of a peptide having an intramolecular cross-linkage via a photocleavable cross-linking group according to the present invention.

FIG. 2 is a view showing one example of a peptide according to the present invention containing a peptide having a protein recognizing site at a part having a cyclic structure, and having a membrane-permeable peptide added thereto.

FIG. 3 is a view showing one example of a peptide according to the present invention containing a peptide having a protein recognizing site at a part having a cyclic structure, and immobilized to a magnetic bead.

FIG. 4 is a view schematically showing one example of a reaction regulation method according to the present invention.

FIG. 5 is a chart showing a mass spectrometry result of a peptide having an intramolecular cross-linkage obtained in Example.

FIG. 6 is a view showing a result of SDS-PAGE for the following samples: an uncross-linked peptide (RLP1-2C), a peptide having an intramolecular cross-linkage, and a peptide having an intramolecular cross-linkage subjected to UV irradiation, obtained in Examples.

FIG. 7 is a view showing results of CD spectra using Mixture solution (I) and Mixture solution (II) obtained in Examples.

### MODES FOR CARRYING OUT THE INVENTION

First, description will be made for a novel peptide having a cyclic structure of the present invention. A peptide having an intramolecular cross-linkage via a photocleavable cross-linking group of the present invention forms a cyclic structure together with the cross-linking group.

For the formation of a complex as will be described later, it is important to comprise a peptide having an epitope corresponding to a functional protein or a ligand, that is, a protein recognizing site, at a part which forms the cyclic structure. Such a protein recognizing site may be originally present in the peptide, or may be artificially added to the peptide.

In the present specification, the term “protein of interest” is synonymous to a functional protein and a ligand. The term “functional protein” refers to an enzyme, a receptor and the like in a biological body. The term “peptide capable of recognizing a protein” is a peptide having a protein recognizing site, which is interacts with the protein of interest to form a peptide-protein complex.

Examples of the photocleavable cross-linking group of the present invention may include a divalent linking group represented by the following chemical formula (I):

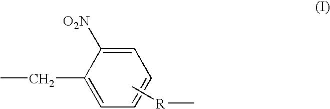

wherein R represents a divalent group.

Concrete examples include a divalent linking group represented by the following chemical formula (II):

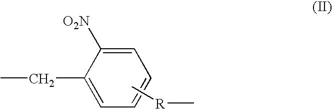

wherein R represents an alkylene group.

As described above, examples of R in the formula may include a bindable divalent organic group, and examples of such a divalent organic group may, for example, include an alkylene group. Examples of the alkylene group may include a methylene group, an ethylene group, a propylene group, a butylene group and the like. The alkylene group may have substituent(s) as appropriate. The alkylene group may occupy a para position of a —CH₂— group in the above formula.

As a photocleavable cross-linking group of the present invention, more concrete examples may include a divalent linking group represented by the following chemical formula (III). Other photocleavable cross-linking groups may be used insofar as they are cleaved by light.

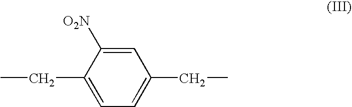

The photocleavable cross-linking group preferably cross-links either between cysteines, between lysines or between cysteine and lysine in a peptide molecule. Cysteine and lysine may be originally present in the peptide molecule or may be artificially added to the peptide.

Next, description will be made for a method for production of a novel peptide having a cyclic structure according to the present invention as described above. In the method for production of a peptide having an intramolecular cross-linkage via a photocleavable cross-linking group according to the present invention, side chain functional groups at two positions in the peptide molecule to be cross-linked and a compound containing the photocleavable cross-linking group are subjected to a cross-linking reaction.

A peptide molecule to be cross-linked which is a starting material may be a naturally occurring molecule, or a synthetic molecule.

Examples of the compound containing the photocleavable cross-linking group used in the cross-linking reaction may include compounds represented by the following chemical formula (IV):

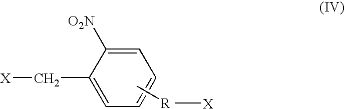

wherein R represents a divalent group and X represents a leaving group or a halogen atom.

Concrete examples may include compounds represented by the following chemical formula (V):

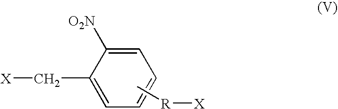

wherein R represents an alkylene group and X represents a leaving group or a halogen atom.

As described above, examples of R in the formula may include a bindable divalent organic group, and examples of such a divalent organic group may, for example, include an alkylene group. Examples of the alkylene group may include a methylene group, an ethylene group, a propylene group, a butylenes group and the like. The alkylene group may have substituent(s) as appropriate.

Examples of the halogen atom may include fluorine (F), chlorine (Cl), bromine (Br), iodine (I) and the like.

Examples of the compound containing a photocleavable cross-linking group used in the cross-linking reaction may more concretely include compounds represented by the following chemical formula (VI):

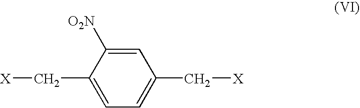

wherein X represents a leaving group or a halogen atom.

One example thereof is 2,5-di(bromomethyl)nitrobenzene (DBMNB) represented by the following chemical formula (VII). Since X is bromine, it is easy to modify an SH group when the photocleavable cross-linking group described later cross-links between SH groups of cysteines.

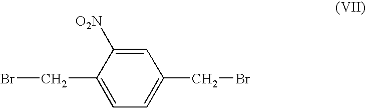

The side chain functional groups at two positions are preferably either of:

an SH group and an SH group of cysteines;

an NH₃⁺ group and an NH₃⁺ group of lysines; and

an SH group of cysteine and an NH₃⁺ group of lysine.

A cross-linking reaction is performed in the manner that is usually performed in the present technical field.

FIG. 1 shows an embodiment example of a method for production of a peptide having an intramolecular cross-linkage via a photocleavable cross-linking group according to the present invention.

In FIG. 1, a chain peptide molecule (1) to be cross-linked reacts with 2,5-di(bromomethyl)nitrobenzene (DBMNB) which is a compound containing a photocleavable cross-linking group, whereby a peptide having an intramolecular cross-linkage via the photocleavable cross-linking group (2) is produced. In FIG. 1, in order for cross-linking between SH groups of cysteines of the peptide molecule, DBMNB having bromine capable of easily modifying the SH group is used.

In FIG. 1, SH groups of cysteines at two positions in a peptide molecule react with DBMNB. An SH group of cysteine and a CH₂ group to which Br in DBMNB binds react to each other to produce a peptide having a cross-linked structure as shown in the figure. A cross-linking reaction is performed by a method which is usually used in the present technical field.

In the peptide having a cross-linked structure shown in FIG. 1, the bond between a —CH₂ group adjacent to a nitro group and S is cleaved upon light irradiation, and the cyclic structure is dissociated at one position. A residue of the cross-linking group remaining in the peptide does not prevent the peptide from taking a tertiary structure and does not prevent the formation of a complex with a protein of interest.

A membrane-permeable peptide may be added to a peptide having an intramolecular cross-linkage via a photocleavable cross-linking group according to the present invention. In order to prevent the photocleavable cross-linking group from erroneously cross-linking with a side chain functional group of a membrane-permeable peptide molecule, it is preferred that the membrane-permeable peptide is added after completion of the cross-linking reaction. When there is a chain part other than a part which forms a cyclic structure, the membrane-permeable peptide is preferably added to one end of such a chain part. When there is no chain part other than a part which forms a cyclic structure, the membrane-permeable peptide may be directly added to the part which forms a cyclic structure. By adding the membrane-permeable peptide, it becomes possible to introduce the peptide into a cell.

As a method of introducing a peptide of the present invention into a cell, injection or introduction by electric pulse that is well known in the art may be recited, as well as addition of a membrane-permeable peptide as described above. As will be described later, when a peptide having an intramolecular cross-linkage via a photocleavable cross-linking group of the present invention is immobilized to a nano bead, a method of impelling the peptide utilizing gas pressure or explosive is also recited.

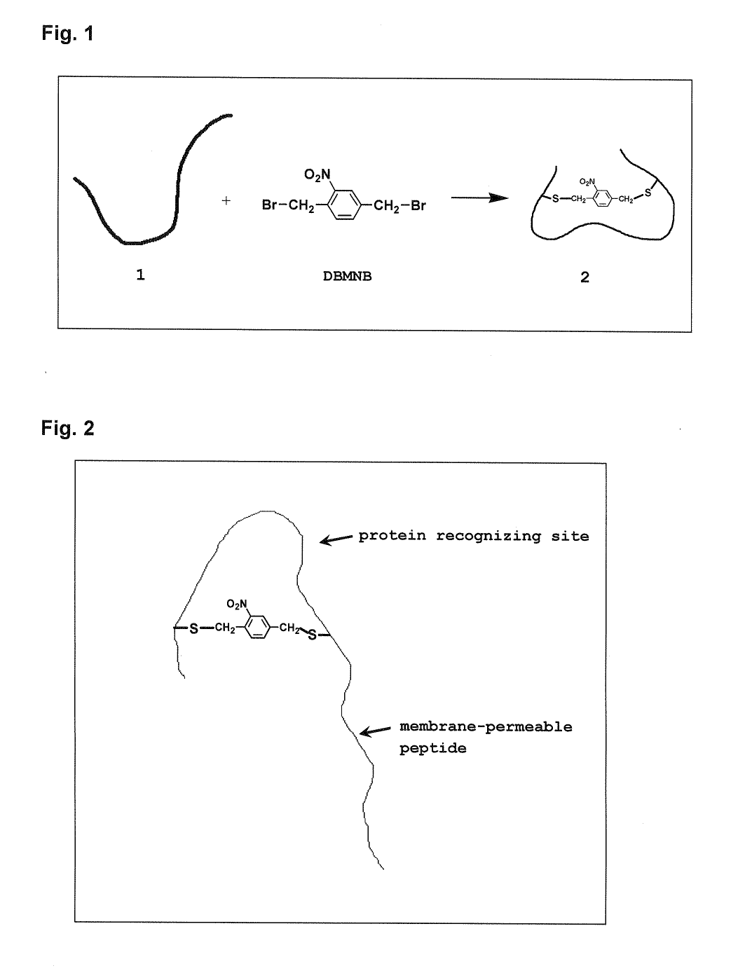

FIG. 2 shows one example of a peptide according to the present invention in which a peptide having a protein recognizing site is comprised in the part having a cyclic structure, and a membrane-permeable peptide is added. In FIG. 2, a membrane-permeable peptide is added to one end of a chain part other than a part which forms a cyclic structure.

The peptide shown in FIG. 2 can be readily taken into a membrane after administration into a body owing to the membrane-permeable peptide added thereto. Upon light irradiation, the photocleavable cross-linking group is cleaved and the cyclic structure is dissociated, so that the peptide is allowed to take a tertiary structure for recognizing a protein, and is able to interact with a protein of interest in the membrane to form a complex therewith.

The peptide having an intramolecular cross-linkage via a photocleavable cross-linking group of the present invention may be immobilized to a nano bead at one end of the peptide. When there is a chain part other than a part which forms a cyclic structure, the nano bead is added to one end of the chain part. When there is no chain part other than a part which forms a cyclic structure, it may be directly added to one position of the part which forms a cyclic structure.

As described above, when a membrane-permeable peptide is added to a peptide having an intramolecular cross-linkage via a photocleavable cross-linking group, immobilization to the nano bead may be achieved via the membrane-permeable peptide.

The nano bead may be a magnetic bead. The peptide which is immobilized to a magnetic bead may be collected by a magnet when administered into a body.

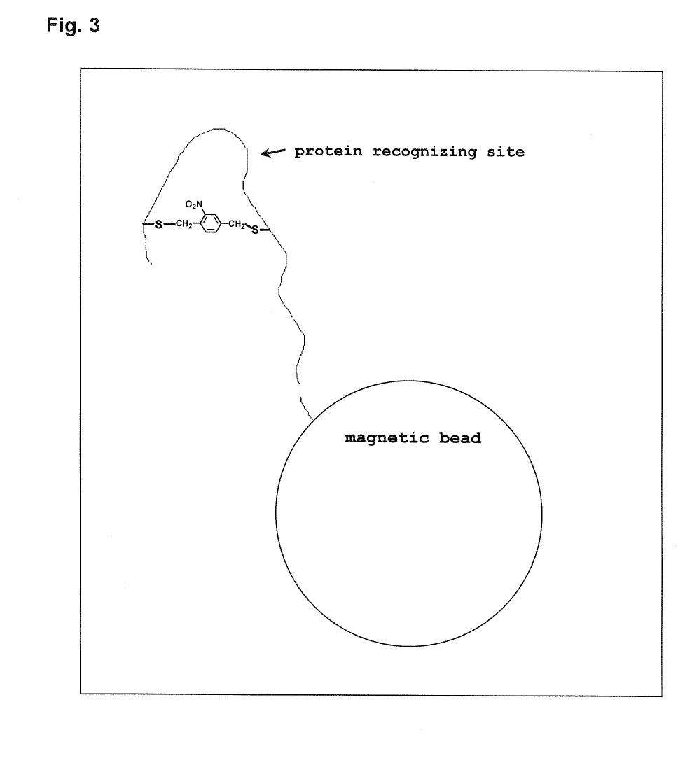

FIG. 3 shows one example of a peptide of the present invention in which a peptide having a protein recognizing site is contained in a region having a cyclic structure, and is immobilized to a magnet bead. In FIG. 3, one end of the peptide having an intramolecular cross-linkage via a photocleavable cross-linking group is immobilized to a magnetic bead. Regarding the peptide shown in FIG. 3, upon light irradiation after being administered into a body, the photocleavable cross-linking group is cleaved and the cyclic structure is dissociated, so that the peptide is allowed to take a tertiary structure for recognizing a protein, and is able to interact with a protein of interest in the membrane to form a complex therewith. Since the formed complex may be collected by a magnet, it is possible to analyze the collected product.

Next, description will be made of a reaction regulation method for formation of a peptide-protein complex using the aforementioned novel peptide having a cyclic structure according to the present invention. The reaction regulation method of the present invention comprises the steps of irradiating a peptide which has an intramolecular cross-linkage via a photocleavable cross-linking group and forms a cyclic structure together with the cross-linking group, and comprises a peptide having an epitope corresponding to a functional protein or a ligand at a part which forms the cyclic structure with light, to cause cleavage of the photocleavable cross-linking group, thereby dissociating the cyclic structure, and initiating a reaction between the peptide in which the cyclic structure has dissociated and the functional protein or the ligand, to form a complex.

As the light, light which has any wavelength capable of causing cleavage of the photocleavable cross-linking group may be used.

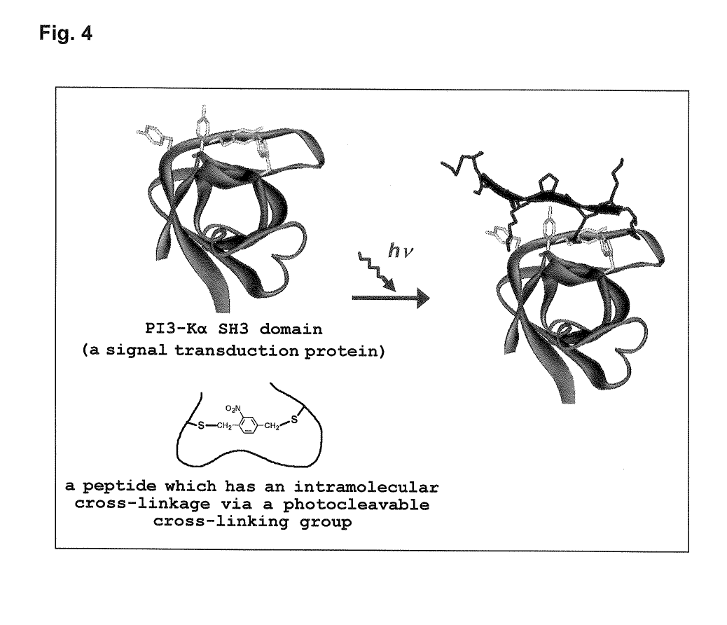

FIG. 4 schematically shows one example of a reaction regulation method of the present invention. In the following, description will be made for a reaction regulation method for formation of a peptide-protein complex according to the present invention, with reference to FIG. 4.

In FIG. 4, a PI3-Kα SH3 domain protein which is widely found in an intracellular signal transduction protein is used as a functional protein. As a peptide having a cyclic structure, a peptide which has a recognition site of a PI3-Kα SH3 domain at a part which forms a cyclic structure and is intramoleculary cross-linked via DBMNB, is used.

The peptide which recognizes a PI3-Kα SH3 domain has a polyproline type II helix structure, however, when it is cross-linked via DBMNB as shown in FIG. 4, it is unable to take free tertiary structure, and does not form a complex even in the presence of a PI3-Kα SH3 domain.

When the cross-linked peptide is irradiated with light, the photocleavable cross-linking group is cleaved, so that the peptide is able to take an original tertiary structure and recognizes the PI3-Kα SH3 domain, to be able to form a complex as is schematically shown in FIG. 4. In the manner as described above, formation of the complex between the functional protein PI3-Kα SH3 domain and the PI3-Kα SH3 domain recognizing a peptide is photoregulated.

Since the peptide which has an intramolecular cross-linkage via a photocleavable cross-linking group, forms a cyclic structure together with the cross-linking group, and comprises a peptide having an epitope corresponding to a functional protein or a ligand at a part which forms the cyclic structure has a cyclic structure, it is not degraded by enzyme even when it is administered into a biological body. Therefore, the formation of a peptide-protein complex may also be photoregulated in a biological body.

As the light, light which has any wavelengths capable of causing cleavage of the photocleavable cross-linking group may be used. In the case of the cross-linking group used herein, UV light is used because of good reaction efficiency of UV light.

Further, description will be made for a method for identifying a complex of the present invention by using a mass spectrometer. The present invention is a method comprising the steps of: immobilizing one end of a peptide which has an intramolecular cross-linkage via a photocleavable cross-linking group and forms a cyclic structure together with the cross-linking group and comprises an epitope corresponding to a functional protein or a ligand at a part which forms the cyclic structure, to a magnetic bead; administering the peptide immobilized to the magnetic bead into a biological body; irradiating the peptide immobilized to the magnetic bead, with light to cause cleavage of the photocleavable cross-linking group, thereby dissociating the cyclic structure, and reacting the peptide in which the cyclic structure has dissociated with the functional protein or the ligand to form a complex; collecting the formed complex by a magnet; and identifying the collected complex by a mass spectrometer.

The peptide may be immobilized to a magnetic bead via a membrane-permeable peptide. By adding the membrane-permeable peptide, the peptide can be taken into the membrane easily when it is administered into a biological body.

Further, a receptor structure is introduced into the part forming the cyclic structure, and the resultant peptide is immobilized to a magnetic bead and administered into a biological body. The peptide having the above receptor structure introduced therein does not interact with a receptor when it encounters the receptor in the biological body, however, upon light irradiation, the photocleavable cross-linking group is cleaved and the cyclic structure is dissociated, so that the peptide is able to interact with a bioactive substance which is reactive with the receptor, and forms a complex. By collecting the formed complex by a magnet, and identifying the collected complex using a mass spectrometer, it is possible to analyze and identify the bioactive substance which is reactive with the receptor.

### EXAMPLES

### 1) Synthesis of Compound DEMNB Containing Photocleavable Cross-Linking Group

Into a 50 mL recovery flask, 0.92 g (5 mmol) of 2,5-di(hydroxymethyl)nitrobenzene, 2.62 g (10 mmol) of triphenylphosphine, and 3.32 g (10 mmol) of carbon tetrabromide were charged and suspended in 20 mL of dehydrated diethylether to give a suspension. Then, the interior of the flask was replaced by nitrogen, and the flask was closely sealed with a septum so as to prevent oxygen and moisture in an atmosphere from entering.

The suspension in the above flask was stirred for 6 hours while the temperature was kept at 25° C. in a thermostat bath, and then stirred for 2 days at room temperature. Solvent in the reaction solution obtained was distilled off under reduced pressure by an evaporator. The residue was dissolved into several tens mL of chloroform, and passed through a column of silica gel (2.5 cm in diameter×3 cm), and all eluate was collected. Further, chloroform for washing (about 200 mL) was passed through a column, and collection was continued until no color is observed in eluate. The collected solution was transferred into a 500 mL recovery flask, and the flask was attached to an evaporator, and chloroform was removed at 40° C. under reduced pressure.

A small amount of the obtained reaction product was taken in an end of a spatula, diluted in about 3 mL of acetone, applied to a thin layer plate, and developed with a mixture solvent of hexane:ethyl acetate=5:1 (volume ratio), and presence/absence of an objective substance was examined under UV light. The objective substance was detected directly above a starting line of the thin layer plate.

The reaction product was dissolved in several tens mL of hexane, and passed through silica gel which is suspended and precipitated in hexane. Then mixed solvent of hexane:ethyl acetate=5:1 (volume ratio) was passed through a column, and the eluate was collected while divided into fractions of about 40 mL, and presence/absence of impurities was examined for each fraction by thin-layer chromatography. Pure fractions were collected until the objective substance was no longer eluted. The collected fractions were transferred into a recovery flask, and solvent was distilled off by an evaporator. The residue was collected and stored in light-shielded condition.

The compound obtained in the manner as described above was subjected to mass spectrometry under the conditions of EI+Mass, JEOL GCmate, chloroform as solvent, and 20 eV, to reveal that the obtained compound was 2,5-di(hydroxymethyl)nitrobenzene (DBMNB) represented by the following chemical formula (VII).

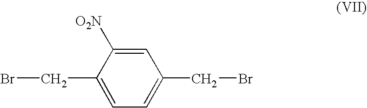

### 2) Cross-Linking Reaction Between Peptide Molecule and DBMNB which is Compound Containing a Photocleavable Cross-Linking Group

A peptide RLP1-2C: Cys Arg Lys Leu Pro Pro Arg Ser Lys Cys (SEQ ID NO: 2 in sequence list) in which cysteine is added to each end of a proline-rich peptide molecule RLP1: Arg Lys Leu Pro Pro Arg Ser Lys (SEQ ID NO: 1 in sequence list) was synthesized by a solid-phase synthetic method of peptide.

RLP1-2C and DBMNB synthesized in the above 1) were subjected to a cross-linking reaction in the following procedure, and an objective peptide having an intramolecular cross-linkage was isolated and purified.

As a pre-treatment, 2-mercaptoethanol was added to RLP1-2C to cause cleavage of an S—S bond, and then 2-mercaptoethanol was removed by dialysis. The pre-treated RLP1-2C (20 mM phosphate buffer solution (KPB), pH 7.0) and DBMNB (dimethylformamide) were mixed in a ratio of 9:1 (volume ratio) to be adjusted so that each concentration of RLP1-2C and DBMNB after mixing was 10 μM. The prepared mixture was subjected to a cross-linking reaction at 50° C. for 40 minutes.

The obtained peptide was adsorbed on a positive-ion exchange column CM52. The column was washed with 10 mM phosphate buffer solution (pH 7.0) to remove excess DBMNB and the like. A peptide was eluted with 3M NaCl dissolved in 10 mM phosphate buffer solution (pH 7.0). Then, the obtained eluate was subjected to gel filtration through a G-25 column. The obtained peptide was dialyzed against ultrapure water (Mili Q water) to remove salt, and then lyophilized.

For the peptide having an intramolecular cross-linkage obtained in the manner as described above, mass spectrometry was performed in the following condition:

Use apparatus: AXIMA-CFR laser ionization time of flight mass spectrometer (manufactured by Shimadzu Corporation)

Extraction voltage: 20 kV

Flight mode: Reflectron

Detected ion: Positive

Matrix: α-cyano-4-hydroxycinnamic acid (CHCA) 10 mg/mL in 0.1% trifluoroacetic acid, 50% acetonitrile (MeCN) saturated solution.

Measurement: Measurements were made three times for the numbers of accumulation times of 10, 50 and 200, while taking resolution by laser beams into account.

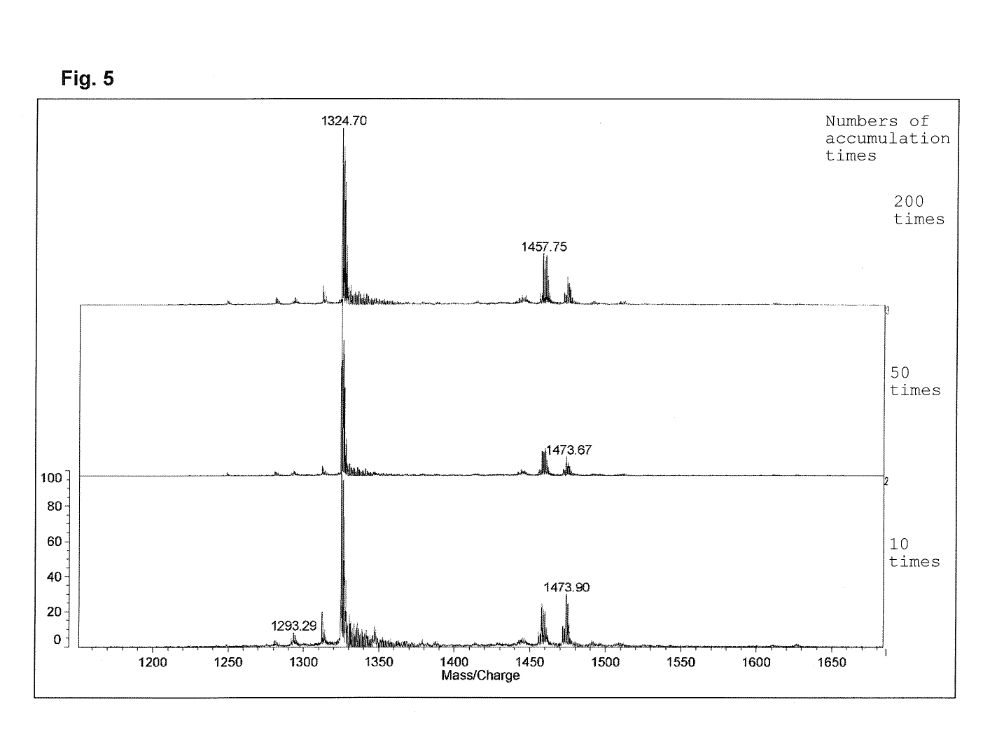

Results are shown in FIG. 5. The horizontal axis of the graph represents Mass/Charge, the vertical axis represents relative intensity of ions. The upper, middle and lower stages in the graph show measurement results for numbers of accumulation times of 200, 50 and 10, respectively.

At a number of accumulation times of 50, observed major peaks were 1248.64, 1280.73, 1293.19, 1311.91, 1324.66, 1433.70, 1457.71, 1459.74, and 1473.67, and the obtained peptide was identified as a peptide wherein RLP1-2C is intramoleculary cross-linked via DBMNB.

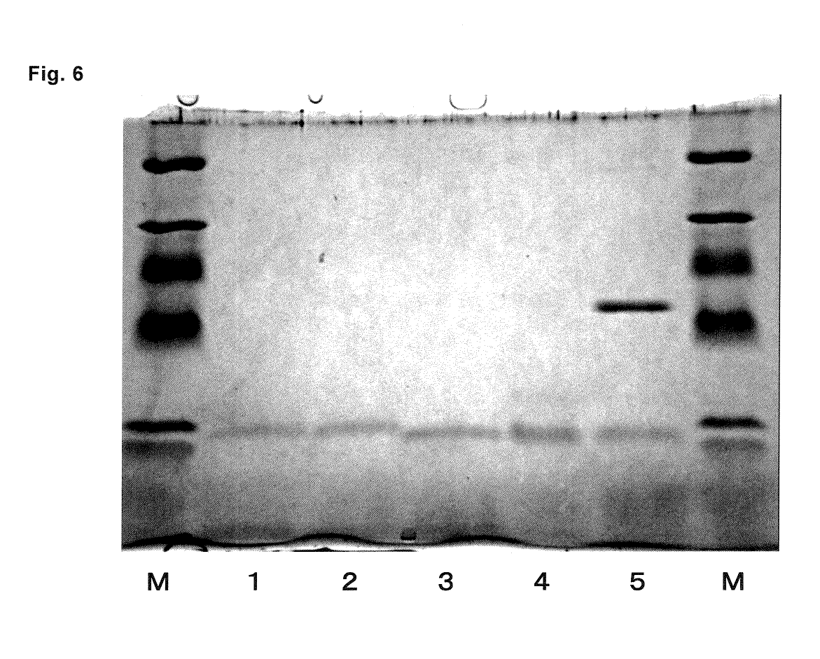

Next, an uncross-linked peptide (RLP1-2C), a peptide having an intramolecular cross-linkage, and a peptide having an intramolecular cross-linkage and irradiated with UV light were subjected to SDS-PAGE. Results are shown in FIG. 6. Lane M is a result of marker, lanes 1 and 2 are results of uncross-linked peptides, lane 3 is a result of a peptide having an intramolecular cross-linkage, and lane 4 is a result of a peptide having an intramolecular cross-linkage and irradiated with UV light. The result of lane 3, seen in comparison with the results of lanes 1 and 2, demonstrates that the peptide having an intramolecular cross-linkage has a cyclic structure, and has compact molecular size.

Lane 5 is a result of a mixture solution containing a peptide having an intramolecular cross-linkage and a later-mentioned PI3-Kα SH3 domain, which is irradiated with UV light.

### 3) Photoregulation of Formation of a Peptide-Protein Complex

As a functional protein, an SH3 domain protein of PI3-Kα was used. The PI3-Kα SH3 domain interacts with RLP1. The peptide having an intramolecular cross-linkage obtained in the manner as described above and the PI3-Kα SH3 domain were mixed in 10 mM phosphate buffer solution (pH 7.0), to obtain Mixture solution (I) in which the concentration of the peptide having an intramolecular cross-linkage was 100 microM and that of the PI3-Kα SH3 domain was 20 microM.

Then, Mixture solution (I) was irradiated with UV light. Irradiation was performed by using Minilite II manufactured by Continuum as a UV irradiator, with pulsed light at wavelength of 355 nm (third harmonic of Nd-YAG laser, pulse width 5 ns, 10 Hz, pulse intensity 4 mJ/cm²) at 4° C. for 30 minutes. By UV irradiation, either one of the bonds between cysteine and DBMNB in the peptide having an intramolecular cross-linkage was cleaved. This was named Mixture solution (II).

A circular Dichroism (CD) spectrum was observed at room temperature for Mixture solution (I) and Mixture solution (II). Used sample solutions are shown below.

(i) Mixture solution (I)
(ii) Mixture solution (II)
(iii) Uncross-linked peptide RLP1 100 microM (10 mM phosphate buffer solution, pH 7.0)
(iv) Peptide having an intramolecular cross-linkage (not irradiated with UV light) 100 microM (10 mM phosphate buffer solution, pH 7.0)
(v) PI3-Kα SH3 domain 20 microM (10 mM phosphate buffer solution, pH 7.0)
(vi) Mixture solution prepared by mixing an uncross-linked peptide RLP1 and a PI3-Kα SH3 domain in 10 mM phosphate buffer solution (pH 7.0), so that the concentration of the uncross-linked peptide is 100 microM and that of the PI3-Kα SH3 domain is 20 microM.

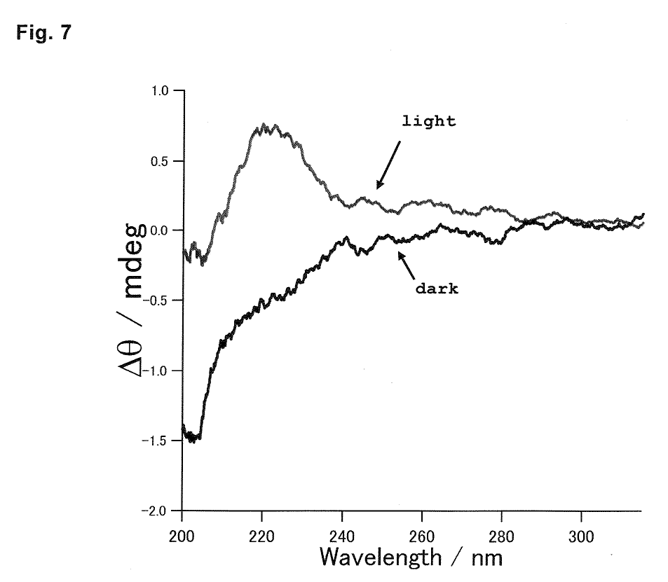

Results are shown in FIG. 7. The horizontal axis of the graph represents wavelength of circularly polarized light (Wavelength/nm), and the vertical axis represents intensity of circular dichroism (Δθ/mdeg). In the graph of FIG. 7, “dark” represents different-al spectrum obtained by subtracting CD spectrum (iv) of the peptide having an intramolecular cross-linkage and CD spectrum (v) of the PI3-Kα SH3 domain from CD spectrum (i) of Mixture solution (I). Further, “light” represents differential spectrum obtained by subtracting CD spectrum (i) of Mixture solution (I) from CD spectrum (ii) of Mixture solution (II).

Positive and negative peaks were not detected in the differential spectrum of dark, and it was demonstrated that there is no interaction between the peptide having an intramolecular cross-linkage and a specific recognizing site of the PI3-Kα SH3 domain. A positive peak was observed around 220 nm in the spectrum of light.

On the other hand, in differential spectrum obtained by subtracting CD spectrum (iii) of the uncross-linked peptide and CD spectrum (v) of the PI3-Kα SH3 domain, from the CD spectrum (vi) of mixture solution of the uncross-linked peptide RLP1 and the PI3-Kα SH3 domain, a positive peak was observed around 220 nm. This peak is ascribable to formation of complex.

From these results, it was confirmed that the cyclic structure of the peptide having an intramolecular cross-linkage is dissociated by UV irradiation, and the peptide in which the cyclic structure has dissociated forms a complex with a PI3-Kα SH3 domain.

1. A peptide having an intramolecular cross-linkage via a photocleavable cross-linking group.

2. A peptide which has an intramolecular cross-linkage via a photocleavable cross-linking group and forms a cyclic structure together with the cross-linking group, containing a peptide having an epitope corresponding to a functional protein or a ligand at a part which forms the cyclic structure.

3. The peptide according to claim 1 or 2, wherein the photocleavable cross-linking group is the following divalent linking group: 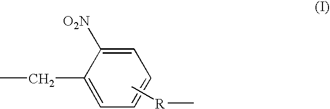
wherein R represents a divalent group.

4. The peptide according to claim 1 or 2, wherein the photocleavable cross-linking group is: 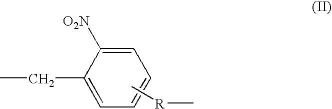
wherein R represents an alkylene group.

5. The peptide according to claim 1 or 2, wherein the photocleavable cross-linking group is:

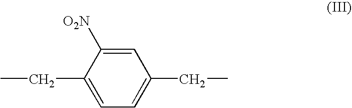

6. The peptide according to claim 1 or 2, wherein the photocleavable cross-linking group cross-links between cysteines, between lysines or between cysteine and lysine in the peptide molecule.

7. The peptide according to claim 1 or 2, wherein a membrane-permeable peptide is added.

8. The peptide according to claim 1 or 2, wherein one end of the peptide is immobilized to a nano bead.

9. The peptide according to claim 8, wherein the nano bead is a magnetic bead.

10. A method for production of the peptide according to claim 1, wherein side chain functional groups at two positions in a peptide molecule to be cross-linked are subjected to a cross-linking reaction with a compound containing a photocleavable cross-linking group.

11. The method for production of the peptide according to claim 10, wherein as the peptide molecule, a peptide molecule containing a peptide having an epitope corresponding to a functional protein or a ligand between the side chain functional groups at the two positions is used.

12. The method for production of the peptide according to claim 10, wherein as the compound containing the photocleavable cross-linking group, 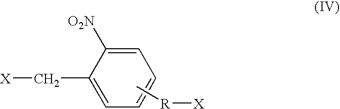
wherein R represents a divalent group and X represents a leaving group or a halogen atom,
is used.

13. The method for production of the peptide according to claim 10, wherein as the compound containing the photocleavable cross-linking group, 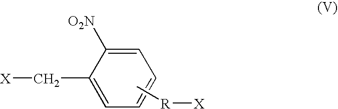
wherein R represents an alkylene group and X represents a leaving group or a halogen atom,
is used.

14. The method for production of the peptide according to claim 10, wherein as the compound containing the photocleavable cross-linking group, 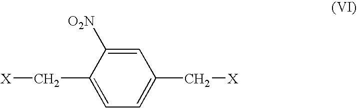
wherein X represents a leaving group or a halogen atom,
is used.

15. The method for production of the peptide according to claim 10, wherein the side chain functional groups at two positions are either of:
an SH group and an SH group of cysteines;
an NH₃⁺ group and an NH₃⁺ group of lysines; and
an SH group of cysteine and an NH₃⁺ group of lysine.

16. The method for production of the peptide according to claim 10, wherein a membrane-permeable peptide is added to the peptide obtained by the cross-linking reaction.

17. The method for production of the peptide according to claim 10, wherein the peptide obtained by the cross-linking reaction is immobilized to a nano bead.

18. The method for production of the peptide according to claim 17, wherein the nano bead is a magnetic bead.

19. A reaction regulation method comprising the steps of:
irradiating a peptide which has an intramolecular cross-linkage via a photocleavable cross-linking group and forms a cyclic structure together with the cross-linking group and comprises a peptide having an epitope corresponding to a functional protein or a ligand at a part which forms the cyclic structure, with light to cause cleavage of the photocleavable cross-linking group, whereby dissociating the cyclic structure; and
initiating a reaction between the peptide in which the cyclic structure has dissociated and the functional protein or the ligand to form a complex.

20. A reaction regulation method comprising the steps of:
administering a peptide which has an intramolecular cross-linkage via a photocleavable cross-linking group and forms a cyclic structure together with the cross-linking group and comprises a peptide having an epitope corresponding to a functional protein or a ligand at a part which forms the cyclic structure, in a biological body;
irradiating the peptide forming the cyclic structure with light to cause cleavage of the photocleavable cross-linking group, whereby dissociating the cyclic structure; and
initiating a reaction between the peptide in which the cyclic structure has dissociated and the functional protein or the ligand to form a complex.

21. The reaction regulation method according to claim 19 or 20, wherein the light for the light irradiation is ultraviolet ray.

22. The reaction regulation method according to claim 19 or 20, wherein the photocleavable cross-linking group is the following divalent linking group: 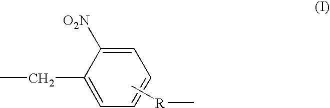
wherein R represents a divalent group.

23. The reaction regulation method according to claim 19 or 20, wherein the photocleavable cross-linking group is: 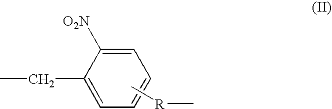
wherein R represents an alkylene group.

24. The reaction regulation method according to claim 19 or 20, wherein the photocleavable cross-linking group is:

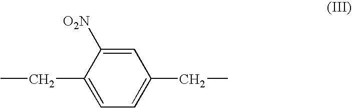

25. The reaction regulation method according to claim 19 or 20, wherein the photocleavable cross-linking group cross-links between cysteines, between lysines or between cysteine and lysine in the peptide molecule.

26. A method comprising the steps of:
immobilizing one end of a peptide which has an intramolecular cross-linkage via a photocleavable cross-linking group and forms a cyclic structure together with the cross-linking group and comprises a peptide having an epitope corresponding to a functional protein or a ligand at a part which forms the cyclic structure, to a magnetic bead;
administering the peptide immobilized to the magnetic bead in a biological body;
irradiating the peptide immobilized to the magnetic bead, with light to cause cleavage of the photocleavable cross-linking group, whereby dissociating the cyclic structure, and reacting the peptide in which the cyclic structure has dissociated with the functional protein or the ligand to form a complex;
collecting the formed complex by a magnet; and
identifying the collected complex by a mass spectrometer.
